(19) United States
(12) Patent Application Publication
(10) Pub. No.: US 2016/0022468 A1
(43) Pub. Date: Jan. 28, 2016

(54) ARM IMMOBILIZATION DEVICE AND ASSOCIATED METHODS

(71) Applicant: Ian K. Y. LO, Calgary (CA)

(72) Inventor: Ian K.Y. Lo, Calgary (CA)

(21) Appl. No.: 14/773,727

(22) PCT Filed: Mar. 6, 2014
(86) PCT No.: PCT/IB2014/000929

§ 371 Date: Sep. 8, 2015

Related U.S. Application Data
(60) Provisional application No. 61/775,097, filed on Mar. 8, 2013.

(51) Int. Cl. A61F 5/37; A61F 5/058

(52) U.S. Cl. CPC A61F 5/373; A61F 5/05858; A61F 5/3753

(57) ABSTRACT

The present disclosure provides exemplary arm immobilization devices for a person and associated methods of use. Thus, in one aspect, the disclosure provides a device including or comprising a belt configured and dimensioned to be detachably secured around a torso of the person. In certain embodiments, the arm immobilization device includes a bolster detachably secured to the belt and configured and dimensioned to support an arm of the person thereon. In another aspect, the disclosure provides methods for immobilizing an arm of a person that include providing an arm immobilization device and supporting the arm of the person with the bolster.

This application claims the benefit of U.S. Provisional Application Ser. No. 61/775,097, filed Mar. 8, 2013, the contents of which is incorporated herein by reference in its entirety.

### TECHNICAL FIELD

The present disclosure relates to arm immobilization devices and associated methods and, in particular, to arm immobilization devices which provide adjustable support to an arm of a person.

### BACKGROUND

Various devices have been used in the medical industry to support an arm of a person after an injury, illness or surgery. For example, slings generally include a sleeve for receiving the arm and a shoulder and/or neck strap which fits around the shoulder or neck of the person to support the weight of the arm. However, the shoulder and/or neck straps of slings generally create a substantial amount of pressure on the shoulder or neck due to the weight of the arm being supported. As a further example, abduction pillows generally provide support for the arm from the side and rely on shoulder, neck and/or upper torso straps to partially support the arm from the bottom. These devices typically do not provide an immobilization of the injured or recovering arm and/or shoulder and can be challenging with respect to adjusting the positioning of the arm.

Thus, a need exists for arm immobilization devices and associated methods which substantially reduce the pressure to the shoulder, neck and/or upper torso of the person and provide convenient means for adjusting the position of the arm being supported. These and other needs are addressed by the devices and methods of the present disclosure.

### SUMMARY

The present disclosure provides exemplary arm immobilization devices for a person and associated methods of use. Thus, in one aspect, the disclosure provides a device including or comprising a belt configured and dimensioned to be detachably secured around a torso of the person. In certain embodiments, the arm immobilization device also includes at least one bolster. In an exemplary embodiment, the bolster is detachably secured to the belt and configured and dimensioned to support an arm of the person. In additional embodiments, the bolster can be further configured and dimensioned to accommodate a brace, e.g., a detachable brace, thereon.

In another exemplary embodiment, the arm immobilization device, as described herein, comprises two bolsters. In certain embodiments, at least one of the bolsters is configured and dimensioned to accommodate a detachable brace as described herein

In any of the embodiments described herein, the bolster and/or the detachable brace can be utilized to at least provide support from below for the arm of the person.

In certain embodiments, the position of the detachable brace relative to the bolster can be adjustable for varying degrees of, e.g., abduction, adduction, internal rotation, external rotation, flexion, extensions, and combinations thereof. In additional embodiments, the belt can be fabricated from an elastic material, e.g., neoprene, and the like. In further embodiments, the bolster can be fabricated from, e.g., a foam material, and the like, and can be configured to be substantially body-contoured to fit against a portion of the torso of the person.

In certain embodiments, the belt includes a fastener, e.g., Velcro®, buttons, adjustable straps, stitching, and the like, for at least one of adjusting sizing of the belt, detachably securing the bolster to the belt, and detachably securing the detachable brace to the bolster. The bolster and the detachable brace, in turn, also include at least one fastener for detachably securing the bolster to the belt and the detachable brace to the bolster, respectively. A position of the bolster relative to the belt can thereby be adjustable along a circumference of the belt and in a vertical direction along the belt.

The detachable brace further includes at least one, and preferably a plurality of fasteners, e.g., adjustable Velcro® straps, adjustable straps with buckles, buttons, clasps, and the like, for releasably securing the arm of the person therein. In certain embodiments, the detachable brace is rigid. In additional embodiments, the detachable brace is formed of a soft padding fabricated from, e.g., elastomeric material, foam material, gel material, rubber material, or the like, including combinations thereof. In still further embodiments, the detachable brace can include a rigid core at least partially surrounded by soft padding. The rigid core can be fabricated from, e.g., a plastic, metal, and the like, to provide a rigid support for the arm.

In some exemplary embodiments, the belt includes a pocket configured and dimensioned to accommodate therein a removable support member. The removable support member can be adapted to provide lumbar support to the person. In an exemplary embodiment, the removable support member can include a rigid core at least partially surrounded by soft padding. The rigid core can be fabricated from, e.g., plastic, metal, and the like, to provide a rigid support for the back of the person. The soft padding can be fabricated from, e.g., a foam material, a gel material, a memory foam material, and the like.

In an additional aspect, the present disclosure provides exemplary methods for immobilizing an arm of a person that include providing an arm immobilization device as described herein. In certain embodiments, the exemplary methods include the step of supporting the arm of the person with the bolster.

In additional embodiments, the exemplary methods include detachably securing a detachable brace to the bolster and adjusting a position of the detachable brace relative to the bolster for varying degrees of, e.g., abduction, adduction, internal rotation, external rotation, flexion, extension, and combinations thereof. In yet additional embodiments, the exemplary methods further include inserting a removable support member into a pocket of the belt. The removable support member can be adapted to provide lumbar support to the person.

Other objects and features will become apparent from the following detailed description considered in conjunction with the accompanying drawings. It is to be understood, however, that the drawings are designed as an illustration only and not as a definition of the limits of this disclosure.

### BRIEF DESCRIPTION OF THE DRAWINGS

To assist those of skill in the art in making and using the disclosed devices and associated methods, reference is made to the accompanying figures, wherein:

FIG. 1 shows a front perspective view of an exemplary arm immobilization device, including an exemplary belt;

FIG. 2 shows a rear perspective view of an exemplary arm immobilization device, including an exemplary belt;

FIG. 3 shows a front view of an exemplary support member;

FIG. 4 shows a rear view of an exemplary support member;

FIG. 5 shows a front perspective view of an exemplary bolster;

FIG. 6 shows a rear perspective view of an exemplary bolster;

FIG. 7 shows a front perspective view of an exemplary arm immobilization device, including an exemplary belt and bolster;

FIG. 8 shows a front perspective view of an exemplary arm immobilization device, including an exemplary belt and bolster;

FIG. 9 shows a side perspective view of an exemplary arm immobilization device, including an exemplary belt and bolster;

FIG. 10 shows a front perspective view of an exemplary arm immobilization device, including an exemplary belt and bolster;

FIG. 11 shows a top view of an exemplary arm immobilization device, including an exemplary belt and bolster;

FIG. 12 shows a perspective view of an exemplary brace;

FIG. 13 shows a perspective view of an exemplary bolster and brace;

FIGS. 14A and 14B show top views of abduction and adduction positioning of an exemplary brace relative to an exemplary bolster;

FIGS. 15A and 15B show top views of internal and external rotation positioning of an exemplary brace relative to an exemplary bolster;

FIGS. 16A and 16B show top views of extension and flexion positioning of an exemplary brace relative to an exemplary bolster;

FIG. 17 is a side view of an exemplary arm immobilization device, including an exemplary belt, bolster and brace, as utilized by a person;

FIG. 18 shows a front view of an exemplary arm immobilization device, including an exemplary belt, bolster and brace, as utilized by a person;

FIG. 19 shows a perspective view of an exemplary arm immobilization device, including an exemplary belt, bolster and brace, as utilized by a person;

FIG. 20 shows a perspective view of an exemplary bolster;

FIG. 21 shows a side perspective view of an exemplary arm immobilization device, including an exemplary belt, bolster and brace;

FIG. 22 shows a side perspective view of an exemplary arm immobilization device, including an exemplary belt, bolster and brace;

FIG. 23 shows a rear perspective view of an exemplary arm immobilization device, including an exemplary belt, bolster and brace;

FIG. 24 shows a front perspective view of an exemplary arm immobilization device, including an exemplary belt, bolster and brace, as utilized by a person;

FIG. 25 shows a rear perspective view of an exemplary arm immobilization device, including an exemplary belt, bolster and brace, as utilized by a person; and

FIG. 26 shows a side perspective view of an exemplary arm immobilization device, including an exemplary belt, bolster and brace, as utilized by a person.

### DESCRIPTION OF EXEMPLARY EMBODIMENTS

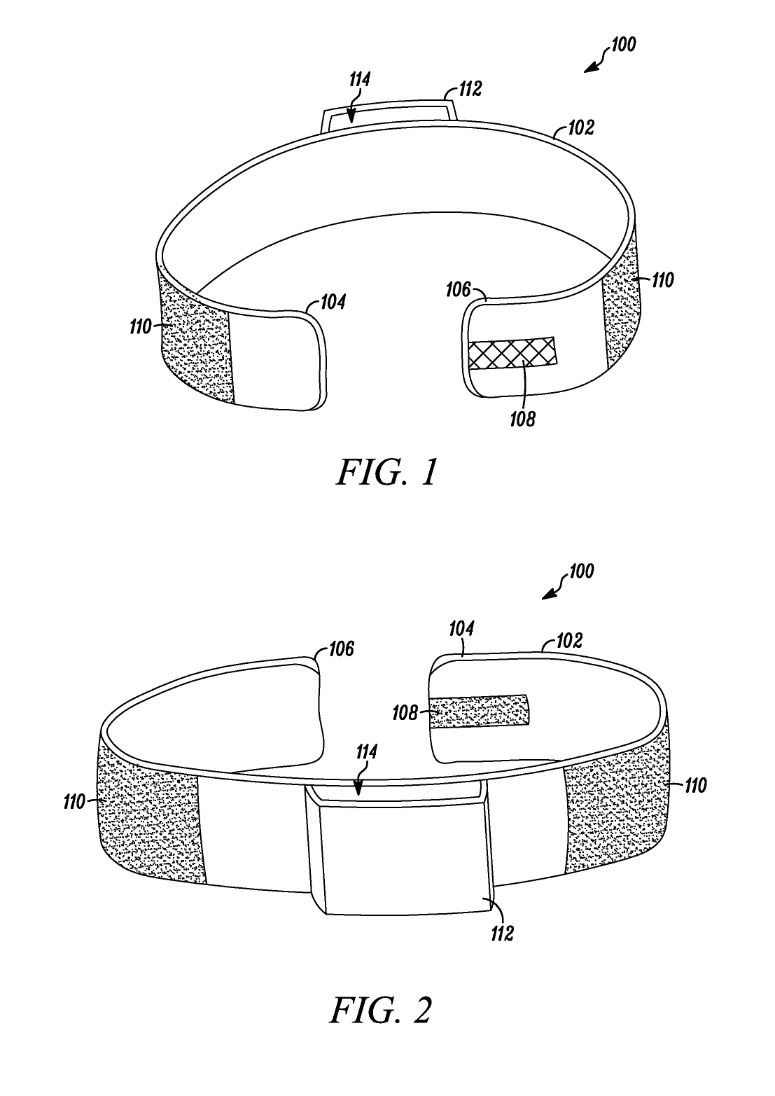

With reference to FIG. 1, a front perspective view of an exemplary arm immobilization device 100 (hereinafter “device 100”) is provided. The device 100 includes a belt 102 configured and dimensioned to be detachably secured around a torso of a person utilizing the belt 102. In particular, the belt 102 includes at least one fastener, e.g., Velcro®, buttons, adjustable straps, and the like, for securing the first end 104 and second end 106 of the belt 102 to each other, thereby detachably securing the belt 102 around the torso of the person. As shown in FIG. 2, exemplary fasteners for the first and second ends 104 and 106 of the belt 102 can be complementary Velcro® strips 108 and/or surfaces. As will be discussed below, the inner surface of the first end 104 and the outer surface of the second end 106 include complementary Velcro® strips 108 such that the first and second ends 104 and 106 can be detachably secured relative to each other. The size of the belt 102 can thereby be adjusted to accommodate a variety of waist sizes, e.g., small, medium, large, and the like. The belt 102 can be fabricated from an elastic material, e.g., neoprene, and the like, to allow the belt 102 to contour the torso of the person wearing the belt 102 and to permit a variability in tension of the belt 102 based on, e.g., the size desired, comfort, adjustment of the lumbar support, and the like. For example, the belt 102 may be tightened by the person such that the belt 102 provides lumbar support.

The exemplary belt 102 further includes at least one fastener, e.g., Velcro® strips and/or surfaces, buttons, stitching, and the like, for detachably securing a bolster to the belt 102. The bolster can then be utilized to support the arm of a person from below. As shown in FIG. 1, the exemplary belt 102 includes Velcro® surfaces 110 along at least a portion of the outer surface of the belt 102. In particular, the belt 102 includes the Velcro® surfaces 110 at least on portions which would be positioned on the left and right side of a person when the belt 102 has been detachably secured around the person's waist. In certain embodiments, the belt 102 includes Velcro® surfaces 110 around the entire circumference of the belt 102. As will be described below, the bolster includes complementary Velcro® surfaces and/or strips for detachably securing the bolster to the belt 102. The bolster can thereby be detachably secured to the left side, right side, or both sides, of the belt 102 to support at least one arm of the person wearing the belt 102.

In some exemplary embodiments, the belt 102 can further include a posterior pocket 112 on an outer surface of the belt 102. The pocket 112 can be positioned along the circumference of the belt 102 such that the pocket rests against the back, e.g., the lower back, of the person when the belt 102 has been secured around a person's torso. The pocket 112 defines an inner area 114 configured and dimensioned to accommodate a removable support member. As will be discussed in greater detail below, the support member can be inserted into the inner area 114 of the pocket 112 to provide lumbar posture support to the person wearing the belt 102. The support member can also, e.g., improve the scapular position of the person, improve the core stability of the person, assist in rehabilitation, and the like. In some exemplary embodiments, the belt 102 itself may be tightened around the waist of the person to provide lumbar support without the utilization of the removable support member.

FIG. 2 shows a rear perspective view of the exemplary device 100 of FIG. 1. As discussed above, the inner surface of the first end 104 of the belt 102 includes a Velcro® strip 108 complementary to the Velcro® strip 108 on the outer surface of the second end 106 (shown in FIG. 1) to detachably secure the first and second ends 104 and 106 relative to each other. FIG. 2 further illustrates the pocket 112 positioned on the rear side of the belt 102.

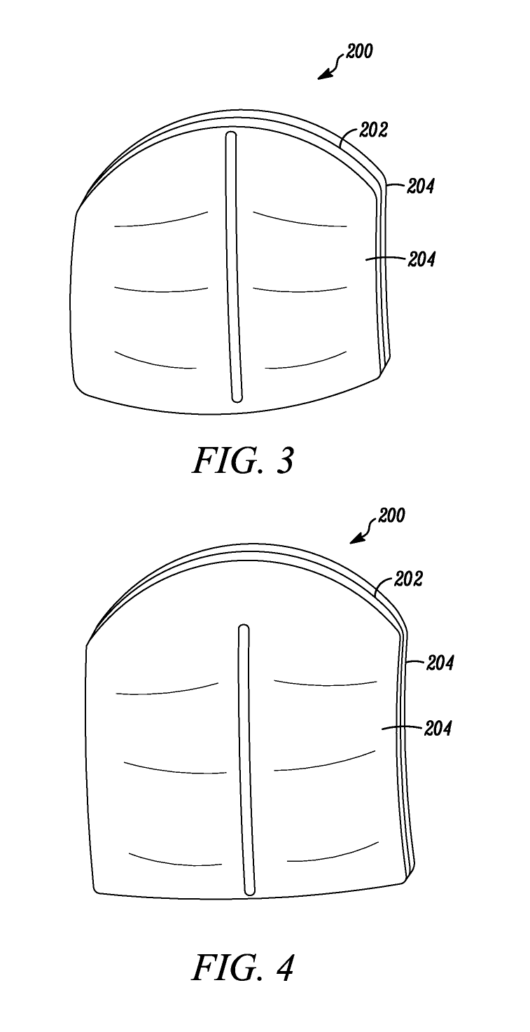

Turning now to FIGS. 3 and 4, front and rear views of an exemplary removable support member 200 are provided. In particular, the support member 200 is configured and dimensioned to be inserted into the inner area 114 of the pocket 112. The support member 200 includes a rigid core 202 fabricated from, e.g., plastic, metal, and the like, at least partially surrounded by a comfort layer 204, e.g., a soft padding, a foam material, a gel material, a memory foam material, and the like. For example, the support member 200 can be a foam reinforced lumbar and/or posture support member. The rigid core 202 provides the stability and/or rigidity necessary for delivering the desired lumbar posture support to the person. The comfort layer 204 provides a soft and/or comfortable surface against which the person's back can be positioned. The desired lumbar posture support can be regulated by tightening or loosening the belt 102. For example, tightening or loosening the belt 102 can increase or decrease the lumbar posture support provided by the support member 200. In addition, tightening or loosening the belt 102 can increase or decrease the lumbar posture support provided by the belt 102 itself.

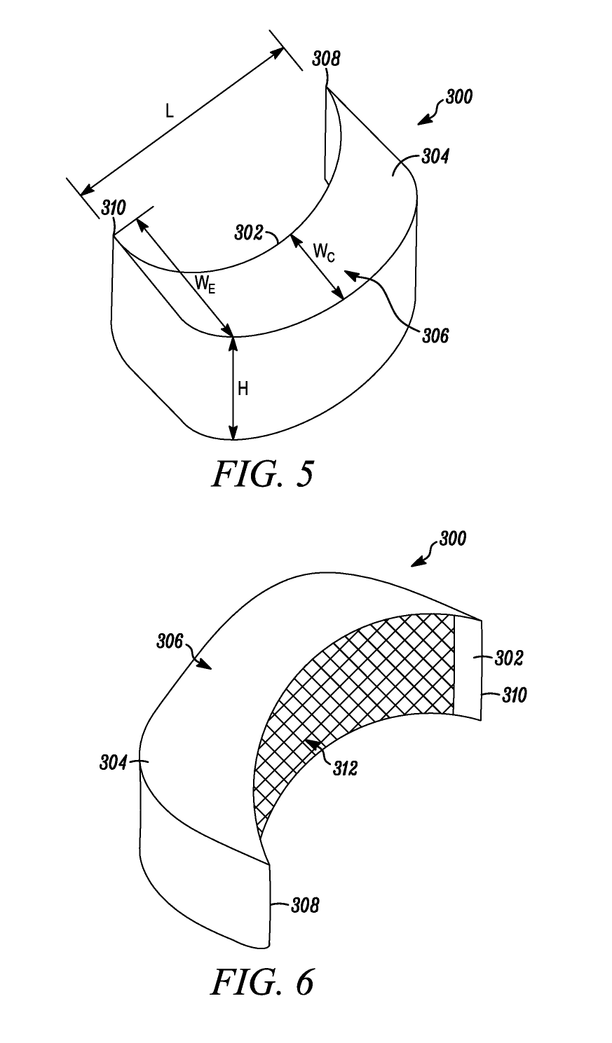

With reference to FIGS. 5 and 6, front and rear perspective views of an exemplary bolster 300 are illustrated. The bolster 300 can be fabricated from a foam material and can be configured as substantially body-contoured to fit against the torso of the person. In particular, the bolster 300 includes a concave inner surface 302 configured as substantially curved to conform to the shape of the torso and a top surface 304 for supporting the arm of the person thereon. In certain embodiments, the height H of the bolster 300 may be less than the height of the belt 100 to allow vertical adjustment of the bolster 300 relative to the belt 100. The bolster 300 may also be covered in a fabric material. The bolster 300 further defines a central body section 306 and first and second edges 308 and 310. The central body section 306 defines a thick section of foam material to provide a semi-rigid surface for supporting the arm of the person thereon. In contrast, the first and second edges 308 and 310 define thinner sections of foam material which permit a flexibility for conforming and/or wrapping the first and second edges 308 and 310 around the torso of the person. For example, the width Wₑ of the central body section 306 can be approximately 15 cm, the width W_{E} of the first and second edges 308 and 310 can be approximately 17 cm, and the length L of the bolster 300 can be approximately 45 cm. Thus, the bolster 300 can be conformed to a variety of torso shapes and sizes.

As discussed above and as shown in FIG. 6, the bolster 300 includes at least one fastener, e.g., Velcro® surface 312, along at least a portion of the inner surface 302 for detachably attaching the bolster 300 to the belt 102. In particular, the Velcro® surface 312 of the bolster 300 is complementary to and mates with the Velcro® surfaces 110 of the belt 102. Thus, one or two bolsters 300 can be detachably secured to the belt 102 for supporting the arm(s) of the person. It should be understood that the bolster 300 can be utilized on both the right and left sides of the belt 102 by detaching the bolster 300 from the belt 102 and attaching it to the appropriate position along the belt 102 circumference. The position of the bolster 300 relative to the belt 102 is further adjustable along the circumference of the belt 102 and in a vertical direction along the belt 102. For example, the bolster 300 can be repositioned along the circumference of the belt 102, i.e., approximately 360° around the belt 102, to position the bolster 300 at the desired angle relative to the arm of the person. As a further example, if a higher or lower support is needed for the arm, e.g., due to differences in length of the arm being supported, the bolster 300 can be vertically raised or lowered, respectively, along the belt 102 to position the bolster 300 at the desired height.

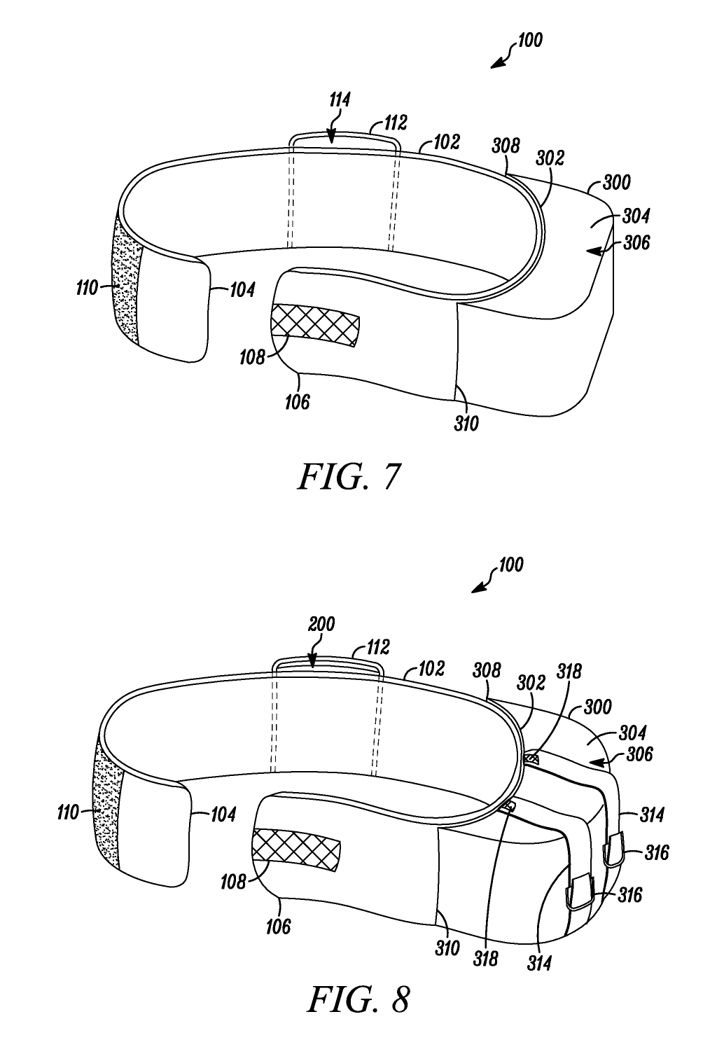

FIG. 7 illustrates the exemplary device 100, including the belt 102 and the bolster 300 detachably secured to the belt 102. As can be seen in FIG. 7, the elastic and/or flexible belt 102 is configured and dimensioned to conform to the torso of the person and the inner surface 302 of the bolster 300 is configured and dimensioned to flexibly conform to the shape of the belt 102 and/or the torso of the person. It should be understood that the fastener between the belt 102 and the bolster 300 is sufficient enough to maintain the connection between the belt 102 and the bolster 300, while providing full support for the arm of the person.

As shown in FIG. 8, in certain embodiments, the device 100 includes vertical straps 314 which are attachable to the belt 102 through fastener 318, e.g., butterfly Velcro® strips, and the like. In particular, the vertical straps 314 can be detachably secured to the belt 102, positioned around the bolster 300, and the length of the vertical straps 314 can be adjusted and/or tightened at, e.g., buckles 316 and/or clasps, to adjust the position of the bolster 300 along a vertical aspect of the belt 102. The vertical straps 314 can further be utilized to ensure that the bolster 300 is securely fastened to the belt 102 and can support the weight of the arm of the person. FIG. 8 also shows the removable support member 200 inserted into the pocket 112 of the belt 102 to provide lumbar support to the person wearing the belt 102. As would be understood by those of ordinary skill in the art, when the belt 102 of FIG. 8 is worn by a person, the bolster 300 (as positioned) would support the left arm of the person and the removable support member 200 and/or the belt 102 would provide lumbar support to the back of the person. However, it should be understood that the bolster 300 can be detached from the belt 102 and repositioned on the right side of the belt 102 to support the right arm of the person.

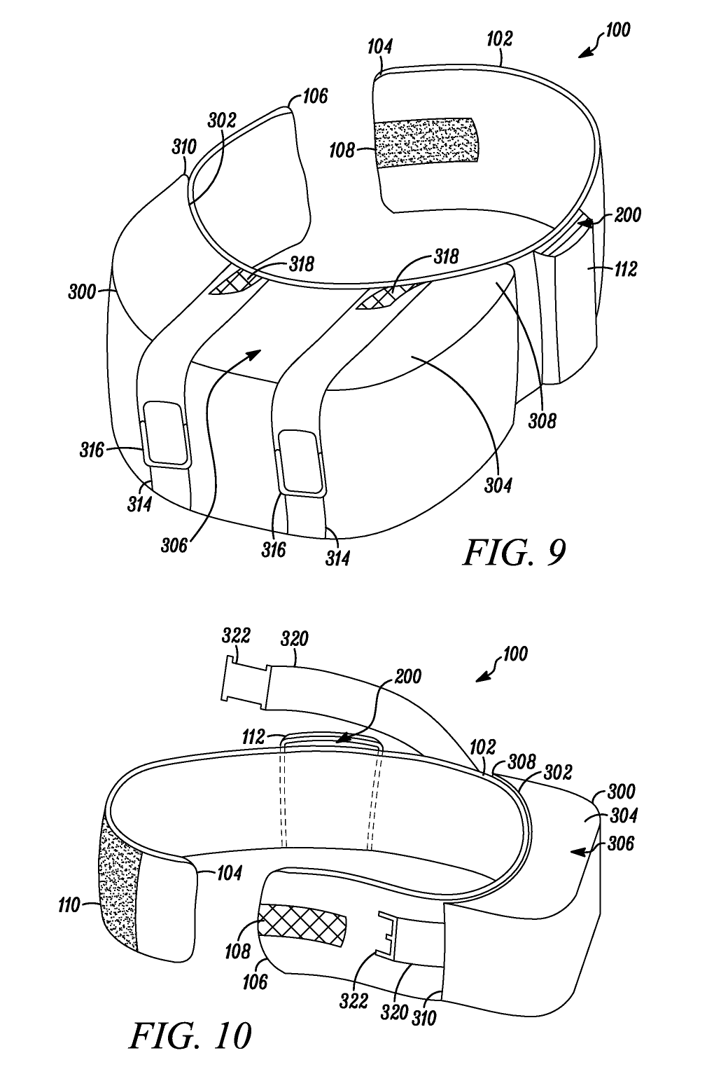

FIG. 9 shows a side perspective view of the exemplary device 100 of FIG. 8. In particular, the device 100 of FIG. 9 includes a belt 102 and a bolster 300 attached to the belt 102. Vertical straps 314 are shown to further support the attachment of the bolster 300 to the belt 102. The removable support member 200 is also inserted into the pocket 112 to provide lumbar support to the person wearing the belt 102. In certain embodiments, if the fastener discussed herein is insufficient to secure the bolster 300 relative to the belt 102, the bolster 300 may be permanently stitched to the belt 102. In this exemplary embodiment, since the bolster 300 would not be removable from the belt 102, the belt 102 could be flipped to switch support between the right and left arms.

With reference to FIG. 10, in some exemplary embodiments, the bolster 300 includes longitudinal strapping for further securing the bolster 300 to the belt 102. The longitudinal strapping can include a strap 320 attached to the first and second edges 308 and 310 of the bolster 300, each strap 320 including complementary male/female components of a fastener 322, e.g., a Delrin® clip buckle and/or fastener, and the like. The strap 320 can include adjustment means to adjust the length of the strap 320 around the circumference of the belt 102. Thus, the belt 102 can be secured around the torso of the person and the straps 320 can be further utilized to tighten the belt around the torso and/or tighten the position of the bolster 300 against the belt 102. The straps 320 may further enhance the lumbar and/or posture support to the person wearing the belt 102 by adjusting the pressure of the removable support member 200 and/or the belt 102 against the back of the person.

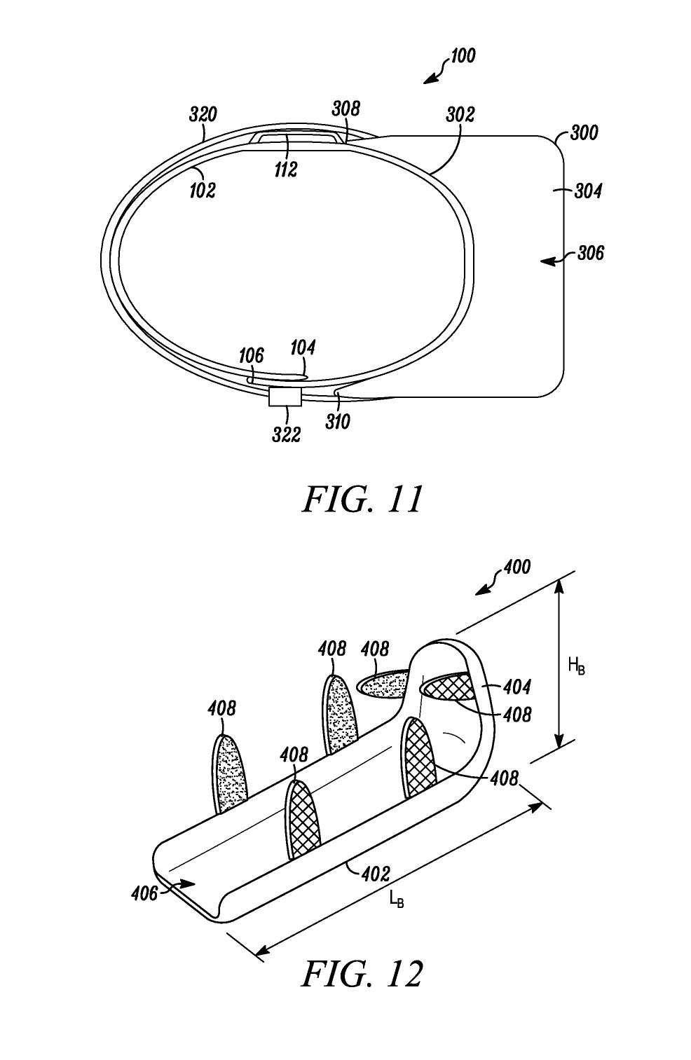

FIG. 11 illustrates a top view of the exemplary device 100, including the belt 102 and the bolster 300. The bolster 300 is attached to the belt 102 and straps 320 with the fastener 322 are utilized to further secure the bolster 300 to the belt 102. As can be seen from FIG. 11, the inner surface 302 of the bolster 300 substantially conforms to the rounded shape of the belt 102, which in turn conforms to the shape of the torso of the person wearing the belt 102.

Turning now to FIG. 12, an exemplary detachable brace 400 is shown. In particular, in some exemplary embodiments, the bolster 300 can be configured and dimensioned to accommodate the brace 400 thereon. For example, the bottom surface 402 of the brace 400 can include at least one fastener, e.g., a Velcro® surface and/or strips, complementary to the at least one fastener on the top surface 304 of the bolster 300. For example, the bolster 300 can be covered in a fabric which mates with the Velcro® surface and/or strips on the bottom surface 402 of the brace 400. Thus, the brace 400 can be detachably positioned on the top surface 304 of the bolster 300 at a variety of desired positions to accommodate the proper support of the arm of the person.

The brace 400 can include a rigid core fabricated from a rigid material, e.g., a plastic, metal, and the like, and further includes a soft material, e.g., a foam material, a gel material, a memory foam material, and the like, at least partially surrounding the rigid core. The rigid material provides the rigidity and/or stability for supporting the arm of the person, while the soft material provides a comfortable surface on which the arm rests. In certain embodiments, the bottom surface 402 and the rear surface 404 are fabricated from the rigid material and an inner surface 406 of the brace 400 is fabricated from the soft material. In particular, the inner surface 406 can be contoured to receive the forearm and/or elbow of the person. It should be understood that the arm of the person can be positioned onto the inner surface 406 of the brace 400, the bottom surface 402 can provide support for the forearm region of the arm, and the rear surface 404 can provide support for the elbow region of the arm, e.g., during supine positioning. In some exemplary embodiments, the length L_{B} of the brace 400 can be approximately 39 cm and the height H_{B} of the rear surface 404 of the brace 400 can be approximately 6 cm. However, it should be understood that the dimensions of the brace 400 can be such that the brace 400 accommodates different arm sizes. In certain embodiments, braces 400 of differing sizes can be provided based on the dimensions of the arm being supported. The brace 400 further includes at least one fastener, e.g., at least one pair of complementary Velcro® straps 408, and the like, for releasably securing the arm, i.e., the forearm, the elbow, the bicep, and the like, of the person against the inner surface 402 of the brace 400. In some exemplary embodiments, as shown in FIG. 12, a plurality of complementary Velcro® straps 408 can be utilized to ensure the arm of the person is tightly secured against the inner surface 402 of the brace 400.

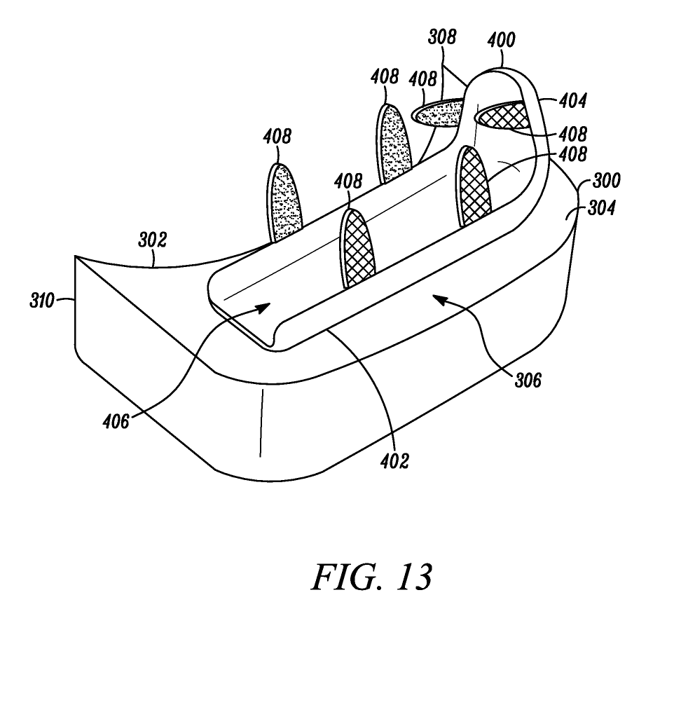

FIG. 13 shows the exemplary brace 400 detachably secured onto the top surface 304 of the bolster 300. The top surface 304 of the bolster can include at least one fastener, e.g., Velcro® surfaces and/or straps, a covering material which mates with the at least one fastener of the brace 400, and the like, along at least a portion of the top surface 304 such that the brace 400 position relative to the bolster 300 can be varied to support the arm in different positions. The brace 400 position can thereby be adapted based on the needs of the person. In certain embodiments, the bolster 300 includes a fastener on the top surface 304 and the bottom surface to allow the person to flip the bolster 300 on either side and maintain the ability to attach the brace 400 thereto. It should be understood that the fastener between the brace 400 and the bolster 300 ensures that once the brace 400 has been attached to the bolster 300 and the arm has been strapped into the brace 400, the arm is maintained in a substantially immobilized position until the straps on the brace 400 have been released.

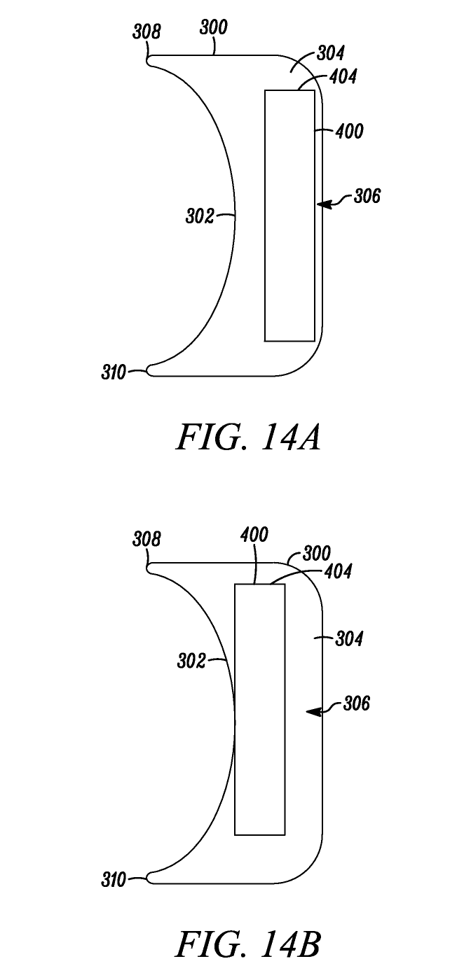

Turning to FIGS. 14A and 14B, top views of the brace 400 positioning relative to the bolster 300 are shown for abduction and adduction. The variability in poisoning the brace 400 on the top surface 304 of the bolster 300 allows control of the position of the forearm and/or shoulder of the person. When viewed from above, abduction and adduction relate to the side-to-side positioning of the brace 400 relative to the bolster 300 along the top surface 304. FIG. 14A shows abduction positioning of the brace 400, i.e., positioning the brace 400 away from the torso of the person. FIG. 14B shows adduction positioning of the brace 400, i.e., positioning the brace 400 closer to the torso of the person.

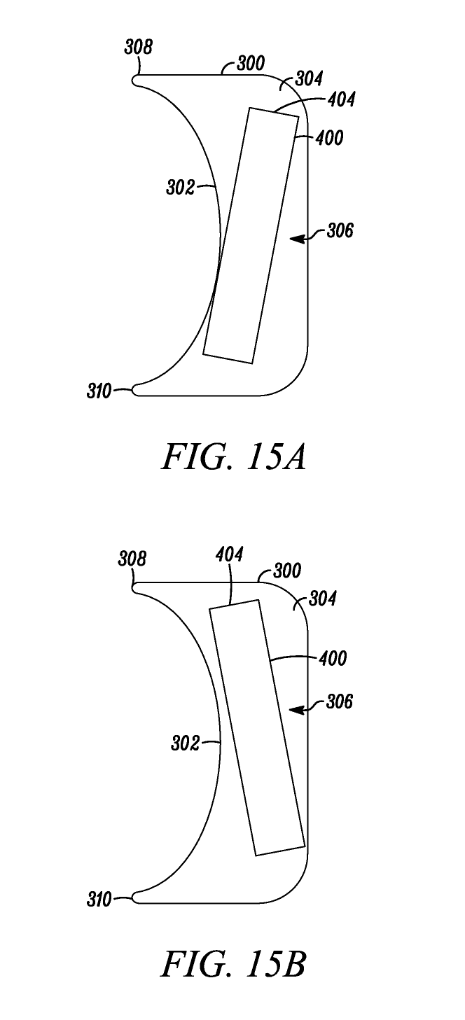

FIGS. 15A and 15B show top views of the brace 400 positioning relative to the bolster 300 for internal and external rotation. When viewed from above, internal and external rotation relate to the clockwise and counterclockwise rotation of the brace 400 relative to the bolster 300 on the top surface 304. FIG. 15A shows an internal rotation of the brace 400, i.e., rotating the brace 400 such that the rear surface 404 of the brace 400 which supports the elbow of the person is rotated away from the torso of the person. FIG. 15B shows an external rotation of the brace 400, i.e., rotating the brace 400 such that the rear surface 404 of the brace 400 which supports the elbow of the person is rotated closer to the torso of the person. Thus, the rotational positioning of the arm of the person can be adjusted as desired.

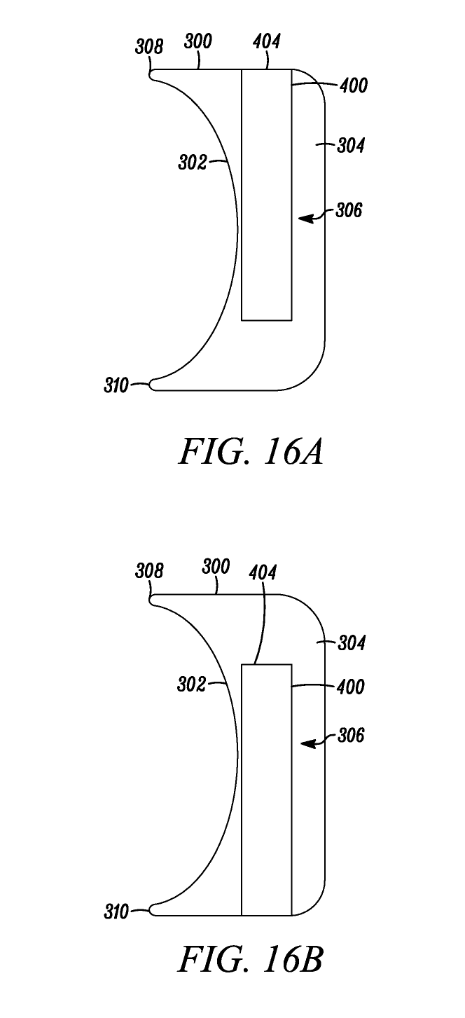

FIGS. 16A and 16B show top views of the brace 400 positioning relative to the bolster 300 for extension and flexion. When viewed from above, extension and flexion relate to the top-to-bottom positioning of the brace 400 relative to the bolster 300 along the top surface 304. FIG. 16A shows an extension position of the brace 400, i.e., positioning the brace 400 such that the rear surface 404 of the brace 400 which supports the elbow of the person is moved posteriorly against the rear portion of the bolster 300. FIG. 16B shows a flexion position of the brace 400, i.e., positioning the brace 400 such that the rear surface 404 of the brace 400 which supports the elbow of the person is moved anteriorly in the direction of the front portion of the bolster 300. Although the adduction, abduction, internal rotation, external rotation, extension, and flexion positioning are shown separately in FIGS. 14A-16B, it should be understood that the position of the brace 400 relative to the bolster 300 can be one or a combination of the types of positions described above to position the forearm of the person in the desired position. It should further be understood that the position of the brace 400 relative to the bolster 300 can be varied as desired by the user by utilizing the entire surface area of the top surface 304 of the bolster 300.

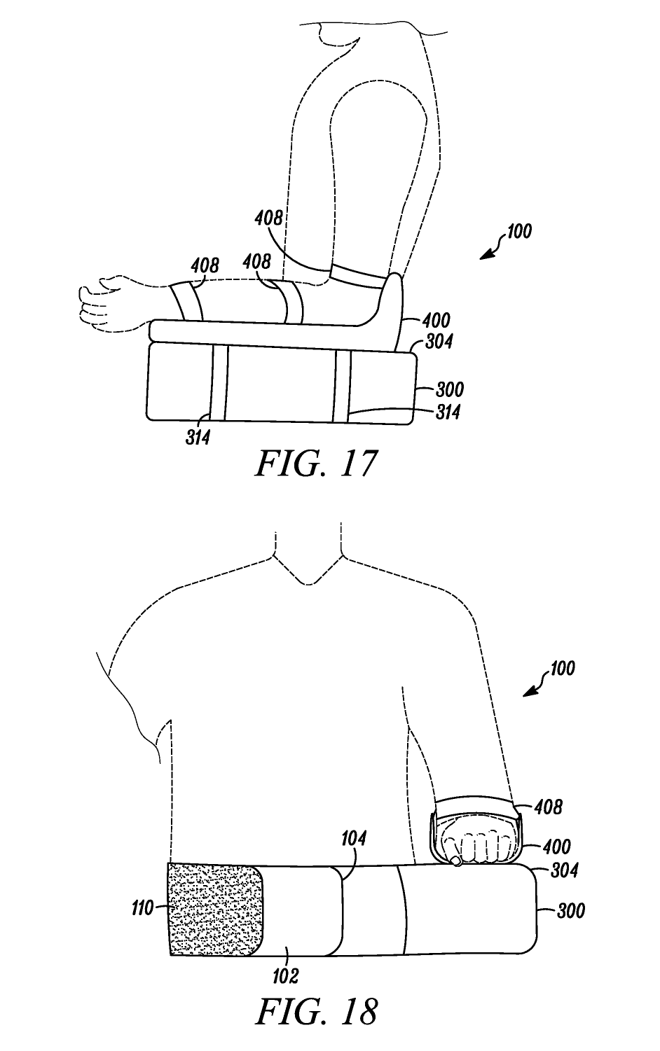

Turning now to FIGS. 17-19, side, front and perspective views of an exemplary device 100 worn by a person to support the left arm are provided. In particular, the belt 102 has been detachably secured around the torso of the person, the bolster 300 has been detachably secured to the belt 102, and the brace 400 has been detachably secured to the top surface 304 of the bolster 300. As can be seen from FIGS. 17-19, the secure fastening of the belt 102 to the torso of the person and, in turn, the secure fastening of the bolster 300 and brace 400 relative to the belt 102 ensure that the arm receives the desired support from below while maintaining a substantially horizontal position of the bolster 300 and/or brace 400. Further, it should be noted that the desired support from below to the arm is provided by the exemplary device 100 without the use of neck, shoulder and/or upper torso straps.

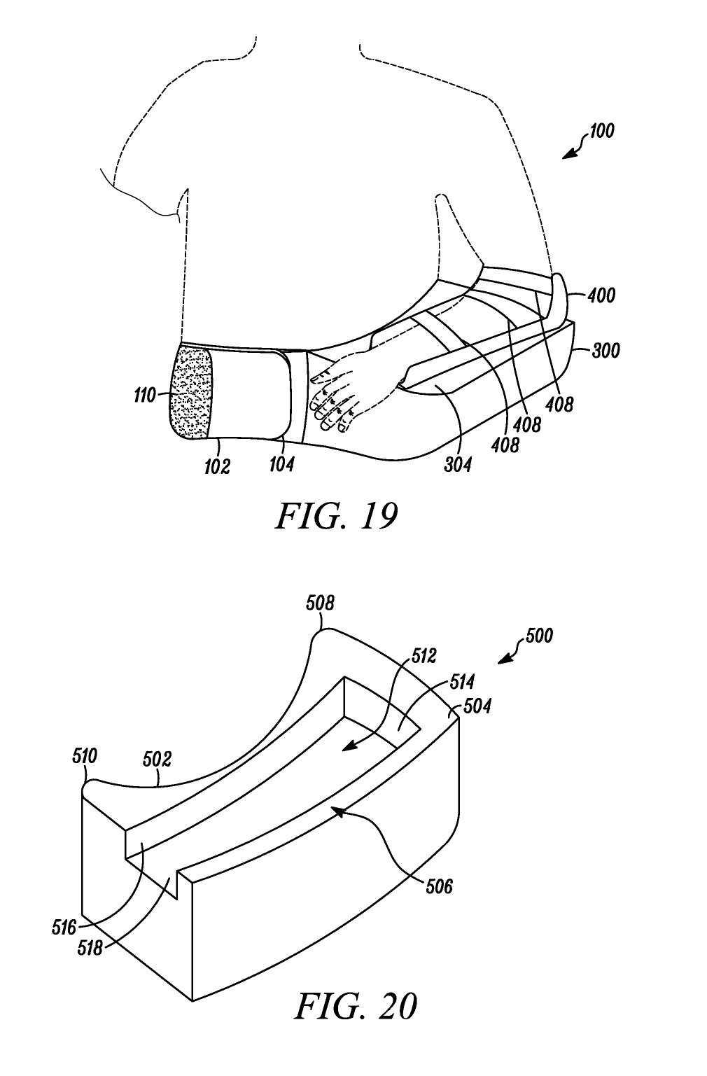

In addition, as described above, FIG. 17 illustrates the implementation of vertical straps 314 for additionally securing the bolster 300 relative to the belt 102. FIG. 18 illustrates the brace 400 positioned relative to the bolster 300 such that there is no internal or external rotation of the brace 400. FIG. 19 illustrates the brace 400 positioned relative to the bolster 300 with an internal rotation of the brace 400 such that the left elbow of the person is rotated away from the body.

With reference to FIG. 20, an alternative embodiment of an exemplary bolster 500 is provided. Similar to the bolster 300, bolster 500 defines an inner surface 502, a top surface 504, a central body section 506, and first and second edges 508 and 510. The inner surface 502 is configured and dimensioned to conform to the contour of the torso of a person. The central body section 506 is fabricated from a thick portion of foam material for providing a semi-rigid surface for supporting the arm of a person thereon. The first and second edges 508 and 510 are fabricated from thinner portions of foam material to flexibly conform to the shape of the torso of the person. The inner surface 502 further includes at least one fastener thereon, e.g., Velcro® surfaces and/or straps, and the like, for detachably fixating the bolster 500 relative to the belt 102.

The exemplary bolster 500 of FIG. 20 further includes a cut-out 512 at the top surface 504 configured and dimensioned to receive the forearm and/or elbow of a person. The cut-out 512 can be defined by a bottom surface 518, side walls 516 and a rear wall 514. The bottom surface 518 can receive and accommodate the forearm of a person, the side walls 516 can provide support to the sides of the forearm, and the rear wall 514 can provide support for the elbow of the person. Thus, rather than utilizing a brace 400, the bolster 500 can be used to support the arm of a person. In certain embodiments, the bolster 500 can include at least one fastener, e.g., at least one pair of complementary Velcro® straps on either side of the cut-out 512 to releasably secure the arm of a person against the bolster 500.

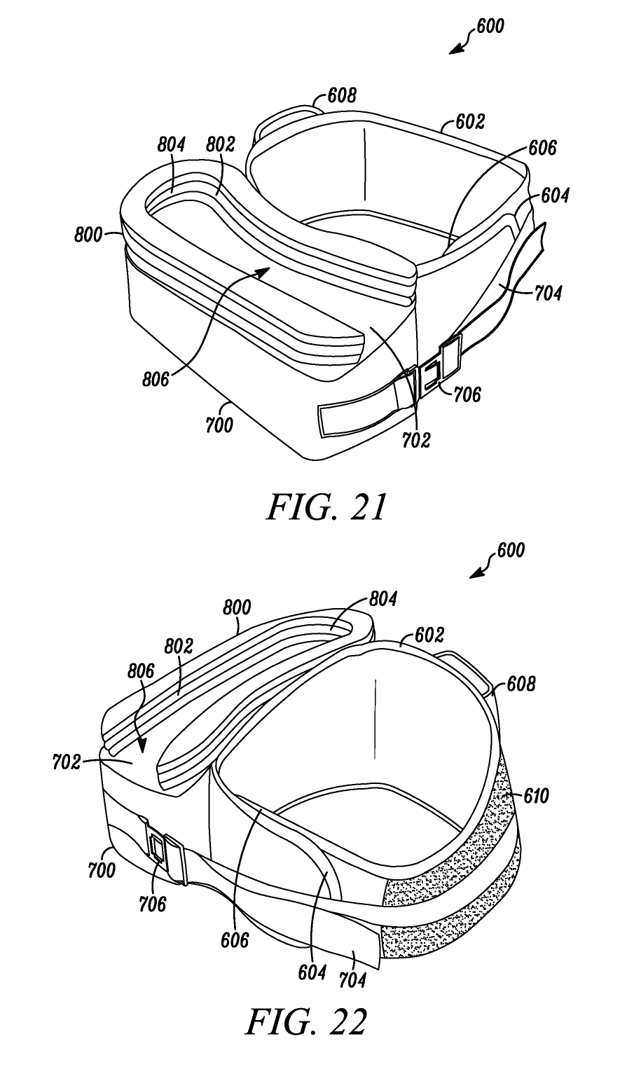

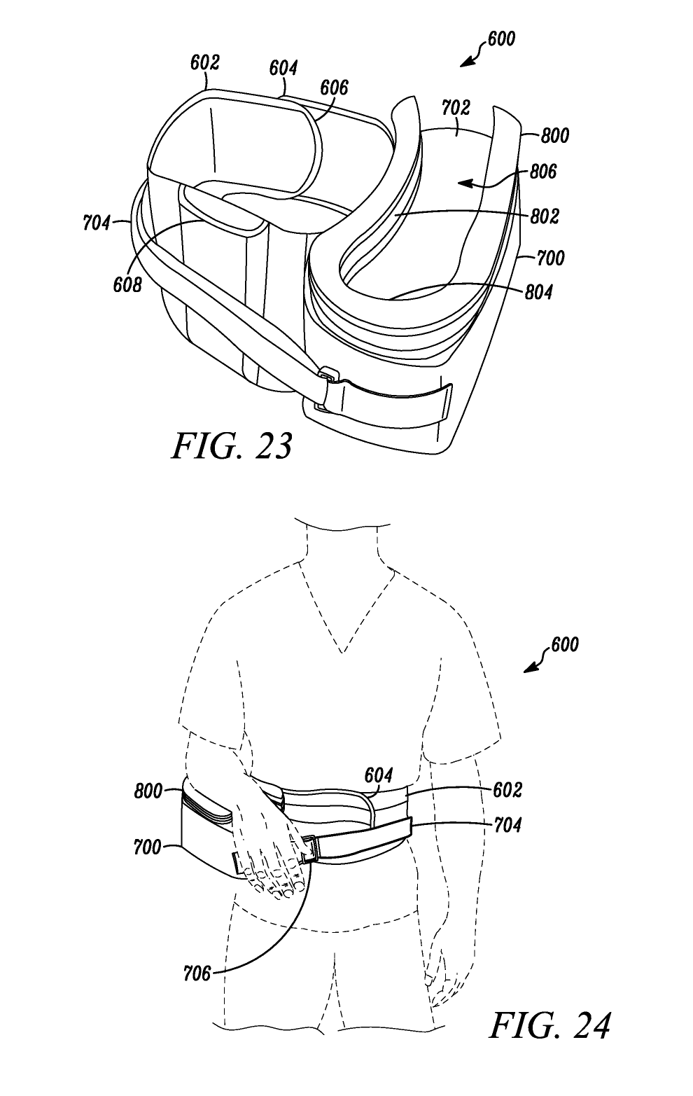

Turning now to FIGS. 21-23, side and rear perspective views of an alternative exemplary arm immobilization device 600 (hereinafter “device 600”) are provided. The exemplary device 600 includes an exemplary belt 602 and bolster 700 substantially similar to the belt 102 and bolster 300 described above. In particular, the belt 602 includes first and second ends 604 and 606 with a fastener (not shown) for detachably securing the first and second ends 604 and 606 relative to each other and around the torso of a person. The belt 602 further includes a pocket 608 for receiving a removable support member therein to provide lumbar support to the person.

The device 600 also includes bolster 700 with at least one fastener on an inner surface complementary to the fastener on the belt 602, e.g., Velcro® surfaces and/or straps 610, and the like, to detachably secure the bolster 700 to the belt 702. The bolster 700 defines a top surface 702 which provides the support to the forearm of a person. The bolster 700 further includes adjustable straps 704 which are configured and dimensioned to surround the belt 602 and attach relative to each other at fastener 706, e.g., a Delrin® clip buckle and/or fastener, and the like.

In some exemplary embodiments, as shown in FIGS. 21-23, the device 600 includes at least one detachable brace 800. The brace 800 can be fabricated from, e.g., a stiff foam material, a soft foam material, and the like. In particular, a plurality of stackable detachable braces 800 can be implemented to vary the depth of the cavity 806 for receiving the forearm and/or elbow of the person. The braces 800 can include at least one fastener, e.g., Velcro° surfaces and/or straps, and the like, on the top and bottom surfaces such that the braces 800 can be detachably secured to the top surface 702 of the bolster 700 and relative to each other. Each brace 800 defines side walls 802 and a rear wall 804. Thus, the top surface 702 of the bolster 700 can provide support from the bottom for the forearm and/or elbow, the side walls 802 can provide support for the sides of the forearm, and the rear wall 804 can provide support for the elbow of the person. It should be understood that if additional support is desired, a greater number of braces 800 can be stacked relative to each other to increase the depth of the cavity 806. Although three braces 800 are shown in FIGS. 21-23, in certain embodiments, e.g., one, two, three, four, five, six, seven, and the like braces 800 can be utilized. In certain embodiments, the bolster 700 can include at least one fastener, e.g., at least one pair of complementary Velcro° straps on either side of the cavity 806 to releasably secure the arm of a person against the bolster 700.

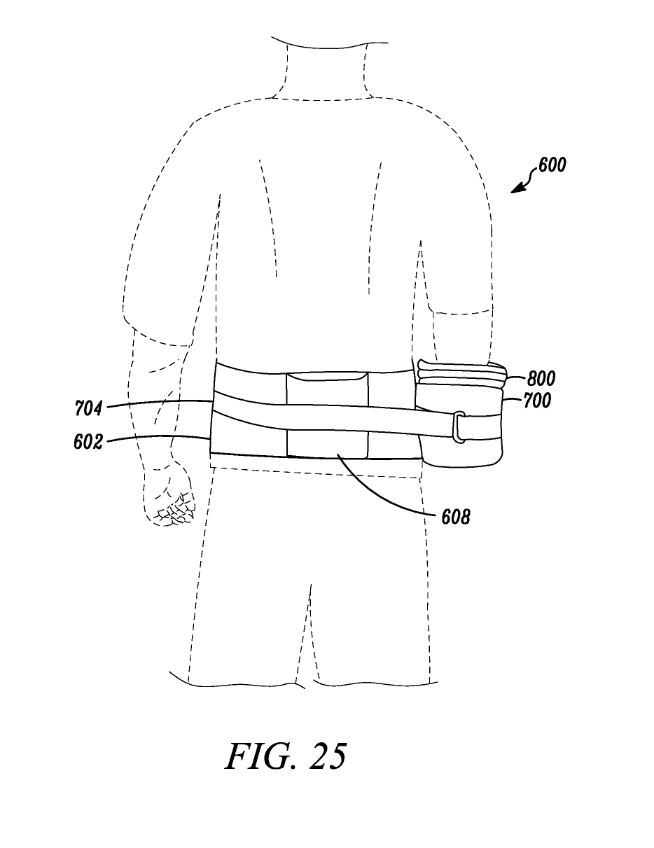

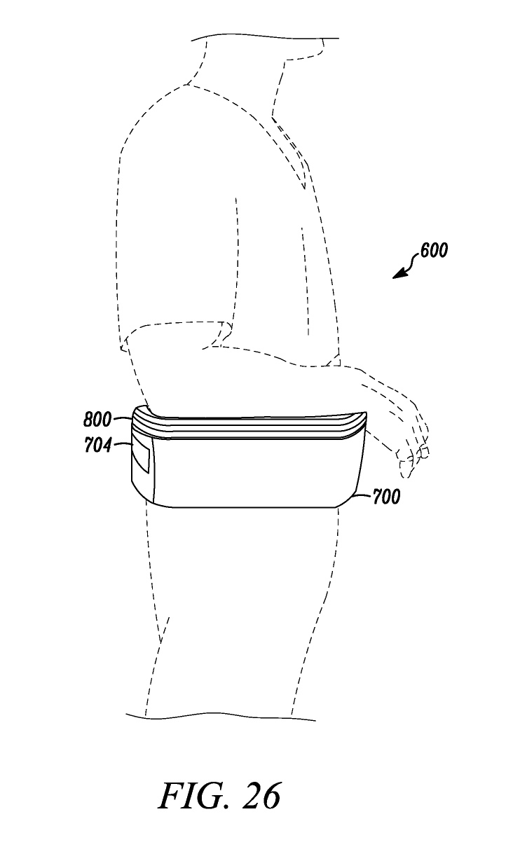

With reference to FIGS. 24-26, front, rear and side views of an exemplary device 600 as worn by a person are provided. In particular, FIGS. 24-26 illustrate the belt 602 detachably secured around the torso of the person and the bolster 700 detachably secured relative to the belt 602 with a fastener between the bolster 700 and the belt 602 (not shown) and with the straps 704 and fastener 706. In addition, FIGS. 24-26 illustrate a plurality of braces 800 detachably secured to the top surface 702 of the bolster 700 to provide support from below and/or from the sides of the arm of the person wearing the device 600. As can be seen from FIGS. 24-26, the exemplary device 600 maintains the arm being supported in a substantially horizontal position relative to the belt 602. As discussed above, in certain embodiments, the bolster 700 can include a fastener, e.g., at least one pair of complementary Velcro® straps on either side of the cavity 806, to releasably secure the arm of the person against the bolster 700 and maintain the arm in a substantially immobilized state. Thus, the exemplary devices discussed herein allow the immobilization of at least one arm of a person, allow a variability in positioning of the immobilized arm, and provide support to the at least one arm from below without the necessity of shoulder, neck and/or upper torso straps.

While exemplary embodiments have been described herein, it is expressly noted that these embodiments should not be construed as limiting, but rather that additions and modifications to what is expressly described herein also are included within the scope of the disclosure. Moreover, it is to be understood that the features of the various embodiments described herein are not mutually exclusive and can exist in various combinations and permutations, even if such combinations or permutations are not made express herein, without departing from the spirit and scope of the disclosure.

1. An arm immobilization device for a person, comprising:
a belt configured and dimensioned to be detachably secured around a torso of the person, and a bolster detachably secured to the belt and configured and dimensioned to support an arm of the person.

2. The arm immobilization device of claim 1, wherein the bolster is configured and dimensioned to accommodate a detachable brace thereon.

3. The arm immobilization device of claim 2, wherein a position of the detachable brace relative to the bolster is adjustable for varying degrees of at least one of abduction, adduction, internal rotation, external rotation, flexion, and extension.

4. The arm immobilization device of claim 1, wherein the belt is fabricated from an elastic material.

5. The arm immobilization device of claim 1, wherein the bolster is configured as substantially body-contoured to fit against the torso of the person.

6. The arm immobilization device of claim 2, wherein the belt includes a fastener thereon for at least one of adjusting sizing of the belt, detachably securing the bolster to the belt, and detachably securing the detachable brace to the bolster.

7. The arm immobilization device of claim 6, wherein the fastener includes at least one of Velcro, buttons, adjustable straps, and stitching.

8. The arm immobilization device of claim 1, wherein a position of the bolster relative to the belt is adjustable along a circumference of the belt and in a vertical direction along the belt.

9. The arm immobilization device of claim 2, wherein the bolster and the detachable brace provide support for the arm of the person.

10. The arm immobilization device of claim 9, wherein the detachable brace includes a plurality of fasteners for releasably securing the arm of the person.

11. The arm immobilization device of claim 10, wherein the plurality of fasteners includes adjustable straps with Velcro.

12. The arm immobilization device of claim 11, wherein the detachable brace includes a rigid core at least partially surrounded by soft padding.

13. The arm immobilization device of claim 12, wherein the rigid core is fabricated from a plastic or metal, and the soft padding is fabricated from at least one of a foam material, a gel material, and a memory foam material.

14. The arm immobilization device of claim 1, wherein the belt includes a pocket configured and dimensioned to accommodate therein a removable support member, the removable support member adapted to provide lumbar support to the person.

15. The arm immobilization device of claim 14, wherein the removable support member includes a rigid core at least partially surrounded by soft padding.

16. The arm immobilization device of claim 15, wherein the rigid core is fabricated from a plastic or metal, and the soft padding is fabricated from at least one of a foam material, a gel material, and a memory foam material.

17. A method for immobilizing an arm of a person, comprising:
providing an arm immobilization device, the arm immobilization device including (i) a belt configured and dimensioned to be detachably secured around a torso of the person, and (ii) a bolster detachably secured to the belt and configured and dimensioned to support the arm of the person, and
supporting the arm of the person with the bolster.

18. The method of claim 17, further comprising detachably securing a detachable brace to the bolster.

19. The method of claim 18, further comprising adjusting a position of the detachable brace relative to the bolster for varying degrees of at least one of abduction, adduction, internal rotation, external rotation, flexion, and extension.

20. The method of claim 17, further comprising inserting a removable support member into a pocket of the belt, the removable support member adapted to provide lumbar support to the person.
